(19) United States
(12) Patent Application Publication
(10) Pub. No.: US 2007/0066824 A1
(43) Pub. Date: Mar. 22, 2007

(54) PREPARATION OF ALFUZOSIN

(76) Inventors: Raghupathi Reddy Anumula, Hyderabad 500 072, A.P. (IN); Sampath Alla, Karim Nagar 505 152, A.P. (IN); Goverdhan Gilla, Hyderabad 500 050, A.P. (IN); Yakambram Bojja, Warangal 506 313, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/534,226

(22) Filed: Sep. 22, 2006

Related U.S. Application Data
(60) Provisional application No. 60/788,758, filed on Apr. 3, 2006.

(30) Foreign Application Priority Data
Sep. 22, 2005 (IN) 1347/CHE/2005

(51) Int. Cl. C07D 405/02

(52) U.S. Cl. 544/284

(57) ABSTRACT

A process for preparing alfuzosin or a salt thereof, which minimizes the concentration of an N¹-(4-amino-6,7-dimethoxyquinazolin-2-yl)-N¹-methyl-N ²-(4-amino-6,7-dimethoxyquinazolin-2-yl)-propane-1,3-diamine impurity in the product.

### INTRODUCTION TO THE INVENTION

The present invention relates to a process for the preparation of alfuzosin and its pharmaceutically acceptable salts. It also relates to a compound related to alfuzosin, herein referred to as “alfuzosin dimer.” In particular, the present invention relates to alfuzosin containing small amounts of alfuzosin dimer and a process for its preparation.

Alfuzosin has the chemical name (R,S)-N-[3-[(4-amino-6,7-dimethoxy-2-quinazolinyl) methylamino] propyl] tetrahydro-2-furancarboxamide and has the structural formula shown as Formula I.

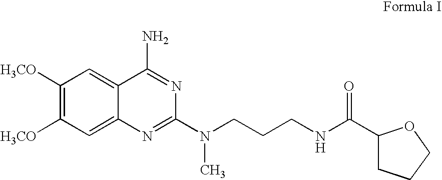

Alfuzosin dimer has the chemical name N¹-(4-amino-6,7-dimethoxyquinazolin-2-yl)-N¹-methyl-N²-(4-amino-6,7-dimethoxyquinazolin-2-yl)-propane-1,3-diamine and has the structural formula shown as Formula II.

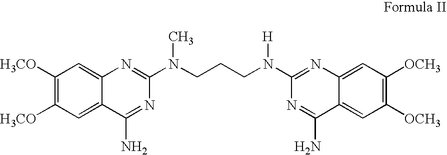

Alfuzosin is an antagonist of α-adrenergic receptors, and is useful as an antihypertensive agent and dysuria treatment agent. It is available in the market from Sanofi-Aventis under the trademark “UROXATRAL” as extended release tablets containing 10 mg alfuzosin hydrochloride as the active ingredient.

U.S. Pat. No. 4,315,007 discloses alfuzosin hydrochloride and its related compounds along with their pharmaceutically acceptable salts and processes for their preparation.

Processes for the preparation of alfuzosin and its pharmaceutically acceptable salts have also been described in U.S. Pat. No. 5,545,738, GB Patent No. 2231571, and Chinese Patent No.1616438. International Application Publication No. 2006/030449 describes a process for the preparation of crystalline alfuzosin free base.

The processes described in the above patents involve the isolation of an unstable ester as an intermediate and also involve multiple stages, which lead to decreased yields and purity of the obtained product. None of the patents have identified the potential impurities obtained in their processes.

Potential impurities in pharmaceutically active agents and formulations containing them include residual amounts of synthetic precursors to the active agent, by-products which arise during synthesis of the active agent, residual solvents, isomers of active agent, excipients used in the preparation of the pharmaceutical formulation, and unidentified adventitious substances. Other impurities which may appear on storage include substances resulting from degradation of the active agent, for instance by oxidation or hydrolysis.

The process for preparing alfuzosin can readily form alfuzosin dimer. However, there is no reference to the alfuzosin dimer in the literature.

The present invention provides a process for the preparation of alfuzosin and its pharmaceutically acceptable salts with a reduced number of synthetic steps, eliminating the need to isolate unstable intermediate. The present invention also identifies one of the potential impurities formed during the synthesis. The process of the present invention can be practiced on an industrial scale, and also can be carried out without sacrifice of overall yield.

### SUMMARY OF THE INVENTION

The present invention relates to alfuzosin or a pharmaceutically acceptable salt thereof containing about 0.003 to about 0.15% of alfuzosin dimer and a process for its preparation.

One aspect of the present invention provides alfuzosin or a pharmaceutically acceptable salt thereof containing about 0.003 to about 0.15% of alfuzosin dimer.

Another aspect of the present invention provides a process for the preparation of alfuzosin or a pharmaceutically acceptable salt thereof containing about 0.003 to about 0.15% of alfuzosin dimer, comprising the steps of:

a) esterifying tetrahydrofuroic acid of Formula III in the presence of an alcohol and an acid to form compound the corresponding ester of Formula IV;

b) condensing the esterified product of Formula IV with 3-methyl amino propylene diamine of Formula V in the presence of a suitable solvent to get N₁-methyl-N₂-tetrahydrofuroyl propylene diamine of Formula VI;

c) condensing N₁-methyl-N₂-tetrahydrofuroyl propylene diamine of Formula VI with 4-amino-2-chloro-6,7-dimethoxyquinozoline of Formula VII to yield alfuzosin free base of Formula VIII;

d) treatment of alfuzosin free base of Formula I with a pharmaceutically acceptable acid in presence of a suitable solvent to afford a pharmaceutically acceptable acid addition salt of alfuzosin represented by Formula VIII.

Suitably, one or more sequential steps are carried out without isolating intermediate compounds. In an embodiment of the invention, step b) is carried out without isolating the intermediate, followed by isolation of the compound of Formula VI.

Yet another aspect of the present invention provides alfuzosin dimer N1-(4-amino-6,7-dimethoxyquinazolin-2-yl)-N1-methyl-N2-(4-amino-6,7dimethoxyquinazolin-2-yl)-propane-1,3-diamine of Formula II.

Still another aspect of the present invention provides pharmaceutical compositions containing a therapeutically effective amount of pure alfuzosin or a pharmaceutically acceptable salt thereof that contains less than about 0.1%, or less than about 0.05%, or less than about 0.01%, or less than about 0.003%, of alfuzosin dimer; along with one or more pharmaceutically acceptable carriers, excipients or diluents.

In an embodiment, a process for preparing alfuzosin or a salt thereof comprises reacting tetrahydro-2-furoic acid with an alcohol to form an ester and, without isolating an ester, reacting with N-methyl-1,3-propanediamine to form N¹-methyl-N²-tetrahydrofuroyl-1,3-propanediamine.

In another embodiment, a process for preparing alfuzosin or a salt thereof comprises reacting N ¹-methyl-N²-tetrahydrofuroyl-1 ,3-propanediamine with 4-amino-2-chloro-6,7-dimethoxyquinazoline and, without isolating an intermediate, reacting with an aqueous base to form alfuzosin.

In a still further embodiment, a process for preparing alfuzosin or a salt thereof comprises:

reacting tetrahydro-2-furoic acid with an alcohol to form an ester and, without isolating an ester, reacting with N-methyl-1,3-propanediamine to form N -methyl-N²-tetrahydrofuroyl-1,3-propanediamine; and

reacting N¹-methyl-N²-tetrahydrofuroyl-1,3-propanediamine with 4-amino-2-chloro-6,7-dimethoxyquinazoline and, without isolating an intermediate, reacting with an aqueous base to form alfuzosin.

In another embodiment, the invention provides alfuzosin or a salt thereof, containing less than about 0.5%, or less than about 0.1%, by high performance liquid chromatography of any of the impurities having the formulae: 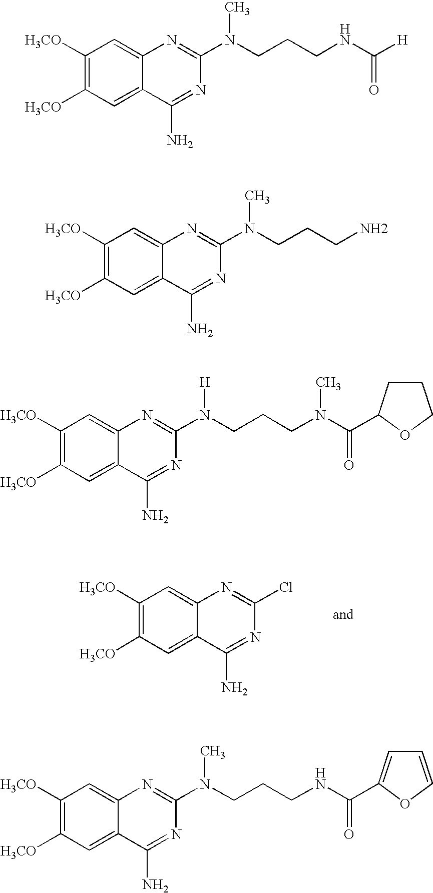
or salts thereof.

### BRIEF DESCRIPTION OF THE DRAWINGS

FIG. 1 is a schematic representation of a process for preparing alfuzosin.

FIG. 2 is a schematic representation of a process for preparing alfuzosin dimer.

### DETAILED DESCRIPTION OF THE INVENTION

In one aspect, the present invention provides alfuzosin containing about 0.003 to about 0.15% of alfuzosin dimer.

Alfuzosin dimer is one of the potential impurities formed during the preparation of alfuzosin, and is carried to the final product which is alfuzosin free base or its pharmaceutically acceptable salts, in the form of an impurity.

Another aspect of the present invention provides a process for the preparation of alfuzosin containing low concentrations of alfuzosin dimer, comprising the steps of:

a) esterifying tetrahydrofuroic acid of Formula III in the presence of an alcohol and an acid to form the corresponding ester of Formula IV; 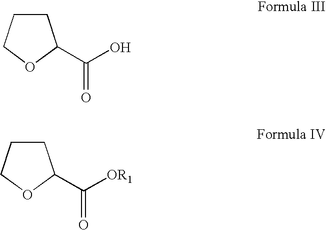
wherein R¹ is a C₁-C₆ straight chain or branched chain alkyl group;

b) condensing the esterified product of Formula IV with 3-methyl amino propylene diamine of Formula V in the presence of a suitable solvent to get N¹-methyl-N²-tetrahydrofuroylpropylenediamine of Formula VI;

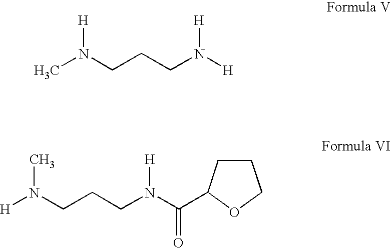

c) condensing the N¹-methyl-N²-tetrahydrofuroylpropylenediamine of Formula VI with 4-amino-2-chloro-6,7-dimethoxyquinozoline of Formula VII to yield alfuzosin free base of Formula I; and

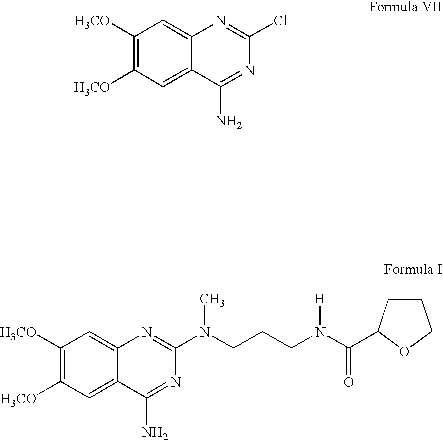

d) treatment of alfuzosin free base of Formula I with a pharmaceutically acceptable acid in presence of a suitable solvent to afford a pharmaceutically acceptable acid addition salt of alfuzosin represented by Formula VIII.

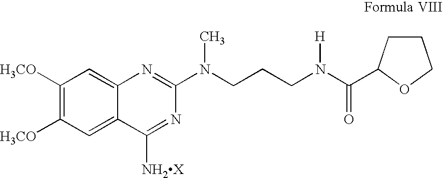

X is a pharmaceutically acceptable acid moiety.

Suitably, one or more sequential steps are carried out without isolating intermediate compounds. In an embodiment of the invention, step b) is carried out without isolating intermediate compound IV, followed by isolation of the compound of Formula VI.

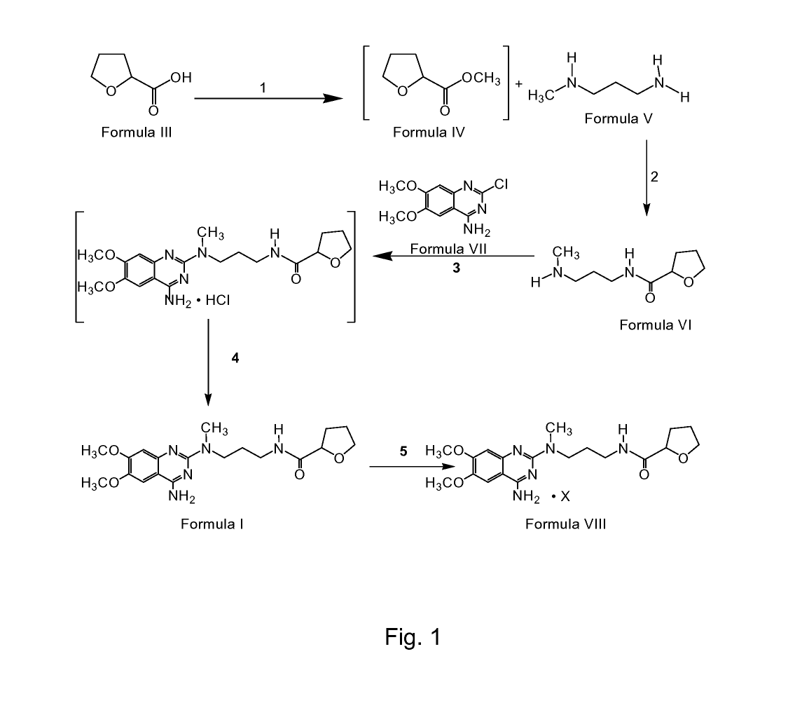

Step a) involves esterification of tetrahydrofuroic acid of Formula III with an alcohol in the presence of an acid to form the corresponding ester of Formula IV. This reaction is Step 1 of FIG. 1.

Suitable alcohols that can be used include, but are not limited to, C₁-C₄ alcohols like methanol, ethanol, butanol, isopropanol, n-propanol, tertiary butanol and the like. The alcohol used for the reaction can be used in stoichiometric excess, such that it functions as a solvent for the reaction.

Suitable pharmaceutically acceptable acids that can be used include, but are not limited to, sulfuric acid, nitric acid, phosphoric acid, acetic acid and the like.

Suitable temperatures for conducting the reaction range from about 10 ° C. to about 50 ° C., or from about 20 ° C. to about 40 ° C.

The ester that is formed as the product has a tendency to convert back to the alcohol when exposed to atmospheric moisture, hence it has to be handled very carefully, if isolated. In an embodiment of the invention, the step is carried out without the isolation of the esterified intermediate product.

Step b) involves condensation of the esterified intermediate of Formula IV with 3-methylaminopropylenediamine of Formula V optionally in the presence of a suitable solvent to get N¹-methyl-N²-tetrahydrofuroylpropylenediamine of Formula VI. This reaction is Step 2 of FIG. 1.

Suitable solvents that can be used for conducting the reaction include, but are not limited to: water; alcohol solvents like methanol, ethanol, isopropyl alcohol and the like; ketone solvents such as acetone, ethyl methyl ketone, methyl isobutyl ketone and the like; hydrocarbon solvents such as toluene, xylene and the like; nitrile solvents such as acetonitrile, propionitrile and the like; and mixtures thereof in various proportions.

Suitable temperatures for conducting the reaction range from about 10 ° C. to about 70 ° C., or from about 30 ° C. to about 50 ° C.

Step c) involves condensation of N¹-methyl-N²-tetrahydrofuroyl propylene diamine of Formula VI with 4-amino-2-chloro-6,7-dimethoxyquinozoline of Formula VII to yield alfuzosin free base of Formula VIII. This reaction is Step 3 of FIG. 1.

The process briefly involves the condensation of N¹-methyl-N²-tetrahydrofuroyl propylenediamine of Formula VI with 4-amino-2-chloro-6,7-dimethoxyquinozoline of Formula VII to yield the hydrochloride salt of alfuzosin. The hydrochloride salt is converted in situ to alfuzosin free base using a suitable base. This reaction is Step 4 of FIG. 1.

Suitable solvents which can be used include, but are not limited to: alcoholic solvents such as methanol, ethanol, isoamyl alcohol, butanol and the like; C₂-C₆ ketone solvents such as acetone, ethyl methyl ketone, and diethyl ketone; chlorinated solvents, such as C₁-C₆ straight chain or branched chlorohydrocarbons including dichloromethane, ethylene dichloride, chloroform, carbon tetrachloride, chlorobenzene, dichlorobenzene and the like; and mixtures thereof.

Suitable temperatures for conducting the condensation reaction range from about 10 ° C. to about 150 ° C., or from about 20 ° C. to about 140 ° C.

Suitable bases which can be used for converting alfuzosin hydrochloride to alfuzosin free base include, but are not limited to: inorganic bases like sodium hydroxide, potassium hydroxide, sodium carbonate, potassium carbonate, sodium bicarbonate and the like; and organic bases like ammonia, dimethylamine, triethylamine, pyridine and the like.

The free base obtained may be optionally purified by recrystallization or slurrying in suitable solvents.

Recrystallization involves providing a solution of crude alfuzosin in a suitable solvent and then crystallizing the solid from the solution.

Suitable solvents in which alfuzosin can be dissolved for purification include but are not limited to: C₁-C₅ ketones such as acetone, ethyl methyl ketone, butanone and the like; alcohols such as ethanol, methanol, and isopropanol; ethers such as such as tetrahydrofuran, 1,4-dioxane, ethyl acetate and the like; water; and mixtures thereof.

The concentration of the alfuzosin in the solvent can range from 40 to 80% or more. The solution can be prepared at an elevated temperature if desired to achieve a higher solute concentration. Any temperature is acceptable for the dissolution as long as a clear solution of the alfuzosin is obtained and is not detrimental to the drug substance chemically or physically. The solution may be brought down to a lower temperature for further processing if required or an elevated temperature may be used. A higher temperature for dissolution will allow the precipitation from solutions with higher concentrations of alfuzosin, resulting in better economies of manufacture.

The product may optionally be further dried. Drying can be suitably carried out in a tray dryer, vacuum oven, air oven, fluidized bed drier, spin flash dryer, flash dryer and the like. The drying can be carried out at temperatures of about 35 ° C. to about 70 ° C. The drying can be carried out for any desired time periods to achieve the desired product purity, times from about 1 to 20 hours frequently being adequate.

Step d) involves treatment of alfuzosin free base of Formula I with a pharmaceutically acceptable acid in presence of a suitable solvent to afford a pharmaceutically acceptable acid addition salt of alfuzosin represented by Formula VIII. This reaction is Step 5 of FIG. 1.

The process briefly involves reacting a pharmaceutically acceptable acid with alfuzosin in solution.

Suitable pharmaceutically acceptable acids which can be used include, but are not limited to: inorganic acids such as hydrochloric acid, hydrobromic acid, hydroiodic acid; and organic acids such as acetic acid, tartaric acid, oxalic acid, and the like.

Optionally, the acid is dissolved in a solvent before adding it to the solution of alfuzosin free base.

Suitable solvents that can be used include, but are not limited to: alcoholic solvents such as methanol, ethanol, isoamyl alcohol, butanol, isopropanol, and the like; C₂-C₆ ketone solvents such as acetone, ethyl methyl ketone, and diethyl ketone; chlorinated solvents, such as C₁-C₆ straight chain or branched chlorohydrocarbons such as dichloromethane, ethylene dichloride, chloroform, carbon tetrachloride, chloro benzene, dichlorobenzene and the like; and mixtures thereof.

The solvent used for the dissolution of alfuzosin free base and the acid may be the same, or different solvents may be used.

Optionally, the acid addition salt obtained can be purified further by recrystallization or slurrying in suitable solvents to make it free from residual and process related impurities.

Suitable solvents in which the acid addition salt of alfuzosin can be dissolved for purification include but are not limited to: C₁-C₅ ketones such as acetone, ethyl methyl ketone, butanone and the like; alcohols such as ethanol, methanol, and isopropanol; ethers such as tetrahydrofuran, 1,4-dioxane, ethyl acetate and the like; water; and mixtures thereof.

The concentration of the acid addition salt of alfuzosin in the solvent can range from 40 to 80% or more. The solution can be prepared at an elevated temperature if desired to achieve a desired concentration. Any temperature is acceptable for the dissolution as long as a clear solution of the alfuzosin is obtained and is not detrimental to the drug substance chemically or physically. The solution may be brought down to a lower temperature for further processing if required or an elevated temperature may be used. A higher temperature will allow the precipitation from solutions with higher concentrations of alfuzosin resulting in better economies of manufacture.

Optionally, small amounts of seeding crystals of alfuzosin acid addition salt will be added to the reaction mixture. Preferably, small amounts are about 1 to 20 weight %, more preferably about 5 weight %. Seeding crystals may be added before or, where appropriate, for initiating the precipitation.

The product may optionally be further dried. Drying can be suitably carried out in a tray dryer, vacuum oven, air oven, fluidized bed drier, spin flash dryer, flash dryer and the like. The drying can be carried out at temperatures of about 35 ° C. to about 90 ° C. The drying can be carried out for any desired time until the required product purity is achieved, time periods from about 1 to 20 hours frequently being sufficient.

The purified alfuzosin obtained above contains about 0.003% to about 0.15% of the alfuzosin dimer impurity of Formula II.

Alfuzosin free base or any of the pharmaceutically acceptable salts of alfuzosin prepared in accordance with the present invention contains less than about 0.5%, or less than about 0.1% of the corresponding impurities like alfuzosin impurity A, impurity B, impurity C, impurity D, or impurity E as characterized by a high performance liquid chromatography (“HPLC”) chromatogram obtained from a mixture comprising the desired compound and one or more of the said impurities. The percentage here refers to weight percent obtained from the area-% of the peaks representing the impurities. Alfuzosin and salts thereof also are substantially free of other process-related impurities

As used herein, “alfuzosin impurity E” refers to N-[3-[(4-amino-6,7-dimethoxyquinazolin-2-yl)(methyl)amino]propyl]formamide represented by Formula IX.

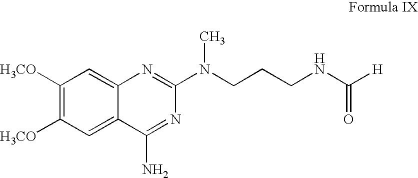

As used herein, “alfuzosin impurity D” refers to N-(4-amino-6,7-dimethoxyquinazolin-2-yl)-N-methylpropane-1,3-diamine represented by Formula X.

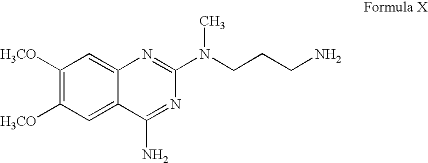

As used herein, “alfuzosin impurity C” refers to (RS)-N-[3-[(4-Amino-6,7-dimethoxyquinazolin-2-yl)amino]propyl]-N-methyltetrahydrofuran-2-carboxamide represented by Formula XI.

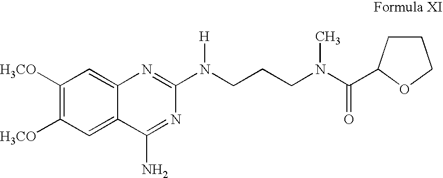

As used herein, “alfuzosin impurity B” refers to 2-chloro-6,7-dimethoxyquinazolin-4-amine represented by Formula VII.

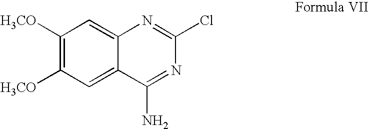

As used herein, “alfuzosin impurity A” refers to N-[3-[(4-amino-6,7-dimethoxyquinazolin-2-yl)(methyl)amino]propyl]furan-2-carbaxamide represented by Formula XII.

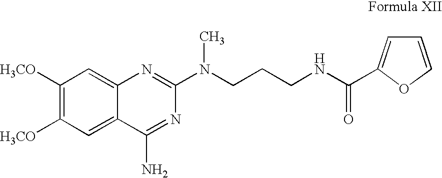

Pharmaceutically acceptable acid addition salts of alfuzosin obtained in this invention contain less than about 5000 ppm, or less than about 3000 ppm, or less than about 1000 ppm, of methanol, and less than about 200 ppm, or less than about 100 ppm, of individual residual organic solvents.

Pharmaceutically acceptable acid addition salts of alfuzosin obtained in this invention contain less than about 3000 ppm, or less than about 2000 ppm, of isopropanol, less than about 200 ppm, or less than about 100 ppm, of methanol, and less than about 10 ppm, or less than about 5 ppm, of acetone and isoamyl alcohol.

Pharmaceutically acceptable acid addition salts of alfuzosin obtained according to the process described in this invention have a bulk density of less than 0.3 g/ml or less than 0.2 g/ml before tapping, and bulk density of less than 0.5 g/ml, or less than 0.2 g/ml after tapping. The bulk densities are determined using Test 616 “Bulk Density and Tapped Density,” United States Pharmacopeia 24, United States Pharmacopeial Convention, Inc., Rockville, Md., 1999, pages 1913-4).

Another aspect of the present invention provides alfuzosin dimer N¹-(4-amino-6,7-dimethoxyquinazolin-2-yl)-N¹-methyl-N²-4-amino-6,7dimethoxyquinazolin-2-yl)-propane-1,3-diamine having structural Formula II.

In order to secure marketing approval for a drug product, a drug manufacturer must submit detailed evidence to the appropriate regulatory authorities to show that the product is suitable for release on to the market. The regulatory authorities must be satisfied, inter alia, that the active agent is acceptable for administration to humans and that the particular formulation which is to be marketed is free from impurities at the time of release and has an appropriate shelf life.

Submissions made to regulatory authorities therefore typically include analytical records, which demonstrate that impurities are absent from the drug at the time of manufacture, or are present only at a negligible level.

These details are usually obtained by testing the drug against an external standard, or reference marker, which is a pure sample of a potential impurity or a potential degradation product. Alfuzosin dimer can be used as a reference marker compound in identifying the purity of the alfuzosin or a pharmaceutically acceptable salt thereof.

It has been identified that alfuzosin dimer is formed as an impurity during the synthesis of alfuzosin or its pharmaceutically acceptable salts.

The formation of alfuzosin dimer impurity in the reaction mass has been identified as result of the reaction of excess 4-amino-2-chloro-6,7-dimethxoyquinazoline of Formula VII with 3-methylaminopropylenediamine of Formula V, and the alfuzosin dimer impurity is carried through beyond Step 3 as an impurity.

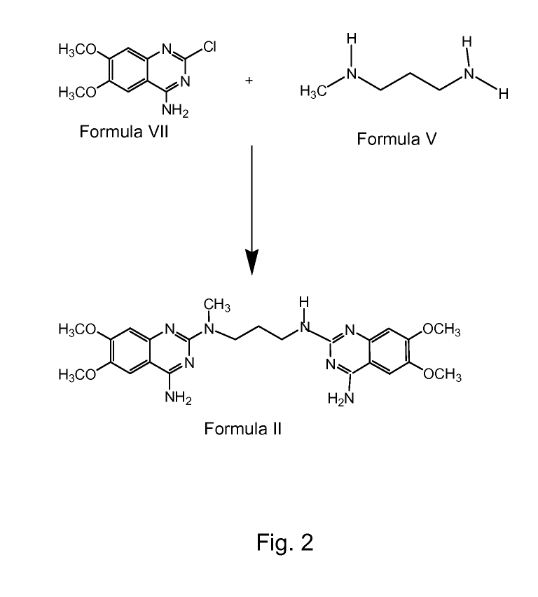

The reaction when reproduced in the presence of the conditions involving reaction of excess of 4-amino-2-chloro-6,7-dimethxoyquinazoline of Formula VII with 3-methylaminopropylenediamine of Formula V in the presence of a suitable solvent gives alfuzosin dimer, as confirmed by its H¹ NMR and mass spectrum. The reaction is shown schematically in FIG. 2.

Suitable solvents, in which the reaction takes place, include, but are not limited to: alcohol solvents such as C₁-C₄ alcohols; C₂-C₆ ketone solvents such as acetone, ethyl methyl ketone, and diethyl ketone; chlorinated solvents, such as C₁-C₆ straight chain or branched chlorohydrocarbons such as dichloromethane, ethylene dichloride, chloroform, carbon tetrachloride, chlorobenzene, dichlorobenzene and the like; and mixtures thereof.

Suitable temperatures for conducting the reaction range from about 10 ° C. to about 50 ° C., or from about 20 ° C. to about 40 ° C.

Still another aspect of the present invention provides pharmaceutical compositions containing a therapeutically effective amount of pure alfuzosin or a pharmaceutically acceptable salt thereof, that contains less than about 0.1%, or less than about 0.003%, of alfuzosin dimer, along with one or more pharmaceutically acceptable carriers, excipients or diluents.

The pharmaceutical composition comprising alfuzosin or its pharmaceutically acceptable salts of the invention along with one or more pharmaceutically acceptable carriers may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir systems or combinations of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinylpyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethylcellulose, methylcellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of the present invention, alfuzosin or its pharmaceutically acceptable salts is a useful active ingredient when present in the range of 0.5 mg to 50 mg, or 1 mg to 25 mg.

Certain specific aspects and embodiments of the present invention will be explained in greater detail in the following examples, which are provided by way of illustration only and should not be construed as limiting the scope of the invention in any manner.

### EXAMPLE 1

### PREPARATION OF N¹-METHYL-N²-TETRAHYDROFUROYLPROPYLENEDIAMINE (FORMULA V)

45 liters of methanol was taken into a reactor and 4.5 kg of tetrahydrofuroic acid was added to it. 0.2 kg of sulfuric acid was added to the above mixture slowly at 28 ° C. The reaction mass was maintained at 30 ° C. for 5 hours. Reaction completion was checked using gas chromatography (“GC”). After the reaction was complete, the mass was transferred into another reactor and another 2 liters of methanol added to it. 3.42 kg of N-methyl-1,3-propanediamine was added to the reactor slowly at 30 ° C., then the mixture was heated to 42 ° C. and maintained for 30 hours. The reaction mass was cooled to 32 ° C. and maintained for 18 hours. Reaction completion was checked using GC. After the reaction was complete, the solvent was distilled off at 65 ° C. under atmospheric pressure. The residue was cooled to 28 ° C. and 24.8 liters of isopropyl alcohol was added to it. The mixture was stirred at 28 ° C. for 45 minutes. The mixture was filtered under a nitrogen atmosphere and the wet cake was washed with 3 liters of isopropyl alcohol. The wet cake was discarded and the combined filtrate was distilled at 65 ° C. under a vacuum of 600 mm Hg. The residue was then cooled to 30 ° C. to yield 6.8 kg of the title compound. (Yield 94.4%)

Purity by GC: 91.47%.

### EXAMPLE 2

### PREPARATION OF PREPARATION OF N¹-(4-AMINO-6,7-DIMETHOXYQUINAZOL-2-YL)-N¹-METHYL-N²-(TETRAHYDROFURONYL-2)-PROPYLENEDIAMINE (FORMUL I)

42 liters of isoamyl alcohol was taken into a reactor and 6.0 kg of 4-amino-2-chloro-6,7-dimethoxyquinozoline was added to it at 27 ° C. 5.59 kg of N¹-(4-amino-6,7-dimethoxyquinazol-2-yl)-N¹ -methyl-N²-(tetrahydrofuronyl-2)-propylenediamine was added to the same reactor at 27 ° C. Another 2 liters of isoamyl alcohol was added to it and the reaction mass was heated to 126 ° C. The mass was maintained at 126 to 128 ° C. for 22 hours. Reaction completion was checked using thin layer chromatography. After the reaction was complete, the mass was cooled to 48 ° C. and was maintained at 48 ° C. for 3 hours and then filtered at 47 ° C. The wet cake was washed with 6 liters of isoamyl alcohol. 90 liters of water was taken into another reactor and the wet cake was added to it. The mixture was stirred for dissolution, and then allowed to settle for separation of the organic layer. The aqueous layer was taken into another reactor and 0.45 kg of carbon was added to it. The mixture was stirred for 45 minutes at 27 ° C. The mixture was filtered through a perlite bed and the bed was washed with 2.25 liters of water. 22.5 liters of acetone was taken into another reactor and cooled to 12.5 ° C. A solution of 16.68 liters of water and 1.67 kg of sodium hydroxide was prepared and it was added to the filtrate kept at 12.5 ° C. The pH of the reaction mass was maintained basic (13). The reaction mass was maintained at the same temperature for 8 hours. The reaction mass was then filtered and the wet cake was washed with a mixture of 1.8 liters of acetone and 7.2 liters of water chilled to a temperature of 12.5 ° C. The mass was filtered to give 8.0 kg of the wet cake. 60 liters of methanol was taken into another reactor and the wet cake was added to it. The mixture was heated to 64 ° C. and maintained for 55 minutes. Then it was cooled to 32 ° C. and maintained for 2 hours. The mixture was filtered and the filter cake was washed with 6 liters of methanol. The wet compound was dried at 77 ° C. under a vacuum of 550 mm Hg for 8 hours to yield 4.5 kg of the tile compound. (Yield 46.29%)

Purity by HPLC: 99.94%.

### EXAMPLE 3

### PREPARATION OF N¹-(4-AMINO-6, 7-DIMETHOXYQUINAZOL-2-YL)-N¹-METHYL-N-²-(TETRAHYDROFUROYL-2)-PROPYLENEDIAMINE HYDROCHLORIDE (FORMULA VIII)

45 liters of isopropyl alcohol was taken into a reactor and 4.5 kg of N¹-(4-amino-6,7-dimethoxyquinazol-2-yl)-N¹-methyl-N²-(tetrahydrofuronyl-2)-propylenediamine obtained above was added to it. 2.34 kg of isopropanolic hydrogen chloride (17.8%) was added to the mixture at 30 ° C. The reaction mass was stirred at 30 ° C. for 1.5 hours. The reaction mass was filtered and the wet cake was washed with 4.5 liters of isopropyl alcohol. The wet cake was dried initially at 57 ° C. and a vacuum of 600 mm Hg for 5 hours and then at 79 ° C. and 600 mm Hg for 18 hours to yield 4 kg of the title compound. (Yield 81.3%)

### EXAMPLE 4

### PURIFICATION OF N¹-(4-AMINO-6,7-DIMETHOXYQUINAZOL-2-YL)-N¹-METHYL-N²-(TETRAHYDROFUROYL-2)-PROPYLENEDIAMINE HYDROCHLORIDE (FORMULA VIII)

35 liters of isopropyl alcohol was taken into a reactor and heated to 64 ° C. 0.18 kg of the title compound was added as seed. In another reactor 22 liters of methanol was taken and 3.5 kg of the material obtained in Example 3 was added to it. The mixture was heated to 64 ° C. and checked for dissolution. The clear solution was added to the first reactor at 62 ° C. 2 liters of methanol was added to it. The solution was distilled at 66 ° C. under a vacuum of 200 mm Hg until the volume of the distillate was 21 liters. Another 18 liters of isopropanol was added to the mass. Another 12 liters of solvent was distilled from the mixture at 65 ° C. and a vacuum of 100 mm Hg. The mass was filtered and the filter cake was washed with 4 liters of isopropanol. The wet compound was dried at 62 ° C. under a vacuum of 600 mm Hg for 4 hours followed by drying at 87 ° C. and a vacuum of 600 mm Hg for 12 hours. The dry material was sifted through a #40 mesh sieve to yield 1.94 kg of the title compound. (Yield 55.43%)

Purity By HPLC: 99.9%.

Dimer impurity: 0.003% by HPLC.

### EXAMPLE 5

### PREPARATION OF N¹-(4-AMINO-6,7-DIMETHOXYQUINAZOLIN-2-YL)-N¹-METHYL-N²-(4-AMINO-6,7-DIMETHOXYQUINAZOLIN-2-YL)-PROPANE-1 3-DIAMINE (FORMULA II)

I) 25 g of 4-amino-2-chloro-6,7-dimethxoyquinazole and 4.6 g of 3-methylaminopropylenediamine were taken into a round bottom flask followed by the addition of 175 ml of isoamyl alcohol and subjected to stirring with simultaneous heating to a temperature of about 130 ° C. for 21 hours. The reaction mass was cooled to a temperature of 26° C., filtered and washed with 25 ml of isoamyl alcohol, and subjected to suction drying followed by oven drying at a temperature of 83 ° C. under a vacuum of 300 mm Hg for 4.5 hours, to afford N¹-(4-amino-6,7-dimethoxyquinazolin-2-yl)-N¹-methyl-N²-(4-amino-6,7-dimethoxyquinazolin-2-yl)-propane-1,3-diamine (Formula II).

II) 30 g of the compound of Formula II was further purified by transferring the compound of Formula II into a round bottom flask followed by the addition of methanol (210 ml), stirring and heating to a temperature of about 50 to 65° C. for about 2.5 hours, then filtering followed by washing with 30 ml of methanol and drying under suction. Finally the compound was dried in an oven at a temperature of about 63° C. under a vacuum of 300 mm Hg for 8 hours.

Purity By HPLC: 96.34%.

Mass (m/z)=494.

¹H NMR (DMSO+CD₃OD) (200 MHz) δ scale: 6.9 (s, 1H), 7.4 (s, 1H), 7.3 (S, 1H), 7.5 (S,1H), 3.5 (m, 2H), 2.0 (m, 2H), 3.1 (m, 2H), 3.8 (s, 3H), 3.88 (s, 3H), 3.82 (s, 3H), 3.9 (s, 3H), 3.2 (s, 3H).

### EXAMPLE 6

### DETERMINATION OF IMPURITIES IN ALFUZOSIN OR ITS HYDROCHLORIDE

TABLE 1
HPLC conditions for determining impurities
in alfuzosin or alfuzosin hydrochloride.

| | |
|---|---|
| Column: | Inertsil ODS-2 150 × 4.6 mm, 5 μ or |
| | equivalent |
| Flow rate: | 1.5 ml/minute |
| Column oven temperature: | 27° C. |
| Detector wavelength: | 254 nm |
| Injection volume: | 20 μl |
| Run time: | 60 minutes |
| Elution: | Isocratic |

| COMPOUND | RRT |
|---|---|
| Alfuzosin impurity D | 0.4 |
| Alfuzosin impurity E | 0.5 |
| Alfuzosin impurity B | 0.6 |
| Alfuzosin dimer impurity | 0.89 |
| Alfuzosin impurity A | 1.2 |
| Alfuzosin | 1 |

We claim:

1. A process for preparing alfuzosin or a salt thereof, comprising reacting tetrahydro-2-furoic acid with an alcohol to form an ester and, without isolating an ester, reacting with N-methyl-1,3-propanediamine to form N¹-methyl-N²-tetrahydrofuroyl-1,3-propanediamine.

2. The process of claim 1, wherein an alcohol comprises methanol in a stoichiometric excess.

3. A process for preparing alfuzosin or a salt thereof, comprising reacting N¹-methyl-N²-tetrahydrofuroyl-1 ,3-propanediamine with 4-amino-2-chloro-6,7-dimethoxyquinazoline and, without isolating an intermediate, reacting with an aqueous base to form alfuzosin.

4. The process of claim 3, wherein reacting N¹-methyl-N²-tetrahydrofuroyl-1,3-propanediamine with 4-amino-2-chloro-6,7-dimethoxyquinazoline occurs in a solvent comprising isoamyl alcohol.

5. The process of claim 3, wherein an aqueous base comprises sodium hydroxide.

6. The process of claim 3, further comprising reacting alfuzosin with hydrogen chloride in isopropanol to form alfuzosin hydrochloride.

7. The process of claim 6, further comprising purifying alfuzosin hydrochloride, by a process comprising combining a solution of alfuzosin hydrochloride in methanol with a dispersion of alfuzosin hydrochloride seed crystals in isopropanol, and removing methanol.

8. The process of claim 7, wherein methanol is removed by distillation.

9. A process for preparing alfuzosin or a salt thereof, comprising:
reacting tetrahydro-2-furoic acid with an alcohol to form an ester and, without isolating an ester, reacting with N-methyl-1,3-propanediamine to form N¹-methyl-N²-tetrahydrofuroyl-1,3-propanediamine; and
reacting N¹-methyl-N²-tetrahydrofuroyl-1,3-propanediamine with 4-amino-2-chloro-6,7-dimethoxyquinazoline and, without isolating an intermediate, reacting with an aqueous base to form alfuzosin.

10. The process of claim 9, wherein reacting N¹-methyl-N²-tetrahydrofuroyl-1,3-propanediamine with 4-amino-2-chloro-6,7-dimethoxyquinazoline occurs in a solvent comprising isoamyl alcohol.

11. The process of claim 9, further comprising reacting alfuzosin with hydrogen chloride in isopropanol to form alfuzosin hydrochloride and purifying alfuzosin hydrochloride by a process comprising combining a solution of alfuzosin hydrochloride in methanol with a dispersion of alfuzosin hydrochloride seed crystals in isopropanol, and removing methanol.

12. The process of claim 11, wherein methanol is removed by distillation.

13. Alfuzosin or a salt thereof, containing about 0.003 to about 0.15% by high performance liquid chromatography of N¹-(4-amino-6,7-dimethoxyquinazolin-2-yl)-N¹-methyl-N²-(4-amino-6,7-dimethoxyquinazolin-2-yl)-propane-1,3-diamine.

14. A pharmaceutical composition comprising a therapeutically effective amount of alfuzosin or a salt thereof of claim 13 and one or more pharmaceutical carriers or excipients.

15. Alfuzosin or a salt thereof of claim 13, containing less than about 0.5 percent by high performance liquid chromatography of any of the impurities having the formulae: 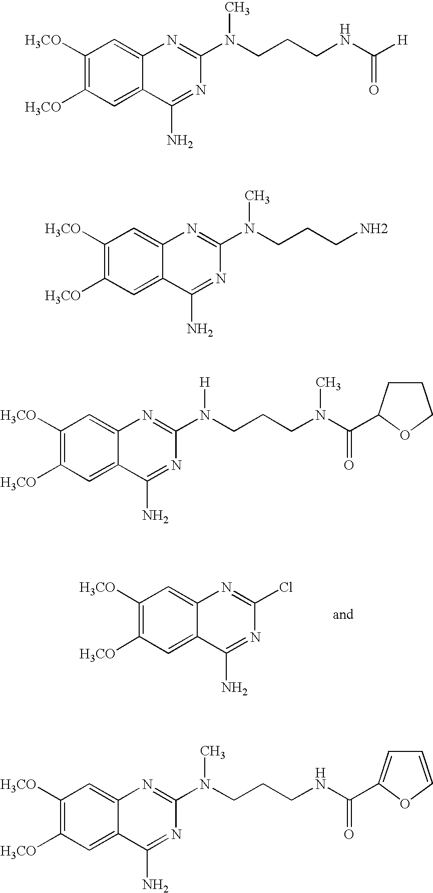
or salts thereof.

16. Alfuzosin or a salt thereof of claim 15, containing less than about 0.1 percent by high performance liquid chromatography of any of the impurities or salts thereof.

17. A pharmaceutical composition comprising a therapeutically effective amount of alfuzosin or a salt thereof of claim 15 and one or more pharmaceutical carriers or excipients.

18. The compound N¹-(4-amino-6,7-dimethoxyquinazolin-2-yl)-N¹-methyl-N²-(4-amino-6,7-dimethoxyquinazolin-2-yl)-propane-1,3-diamine.
